(19) United States
(12) Patent Application Publication
(10) Pub. No.: US 2003/0206914 A1
(43) Pub. Date: Nov. 6, 2003

(54) Synthetic antigens for CD1-restricted immune responses

(75) Inventors: Steven A. Porcelli, Bronx, NY (US); D. Branch Moody, West Roxbury, MA (US)

Correspondence Address: Maria A. Trevisan, Wolf, Greenfield & Sacks, P.C., 600 Atlantic Avenue, Boston, MA, 02210, US

(73) Assignee: The Brigham and Women's Hospital, Inc., Boston, MA

(21) Appl. No.: 10/438,383

(22) Filed: May 15, 2003

Related U.S. Application Data
(63) Continuation of application No. 09/151,869, filed on Sep. 11, 1998.
(60) Provisional application No. 60/058,938, filed on Sep. 12, 1997.

(51) Int. Cl. A61K 39/00

(52) U.S. Cl. 424/184.1

(57) ABSTRACT

Synthetic antigens which comprise hydrophobic and hydrophilic components are provided for inducing CD1-restricted T cell responses in mammals. Further provided are methods for using these antigens and compositions which are combinations of CD1-recognized synthetic antigens, additional antigens, adjuvants and other substances that induce immune responses.

### RELATED APPLICATION

This application claims the benefit of U.S. Provisional Application No. 60/058,938, filed Sep. 12, 1997, the contents of which are incorporated herein by reference in their entirety.

### GOVERNMENT SUPPORT

This invention was supported, in whole or in part, by grants NIH/NIAMS grant AR01988, NIH grants GM54045 and RR10888, NAIAD/NIH grants AI18357 and AI38087, and NIH/NIAID grant AI40135. The United States Government has certain rights in the invention.

### BACKGROUND OF THE INVENTION

T lymphocytes represent an important component of the immune response against a variety of pathogens and tumors. Some T cells are activated directly by antigens while other types of T cells only recognize antigens which are presented by molecules on antigen-presenting cells (APCs).

Until recently, only major histocompatibility complex (MHC) class I or class II-type molecules were known to bind and present antigen fragments to T cells. However, it is now known that another class of proteins, CD1 proteins, plays a role in antigen presentation to T cells in a restricted manner to induce a long term memory T cell response. Unlike MHC class I or MHC class II molecules which only present peptide antigens, CD1 proteins present non-peptide antigens to T cells.

CD1 proteins play a central role in the specific T cell recognition of lipid and glycolipid antigens, but the molecular and structural mechanisms underlying lipid or glycolipid antigen presentation are not known. CD1 proteins represent a family of nonpolymorphic molecules which are encoded by five nonpolymorphic CD1 genes in humans (CD1a, CD1b, CD1c, CD1d and CD1e), four of which are known to be expressed on antigen-presenting cells such as Langerhans cells, dermal and lymph node dendritic cells, mantle zone B cells and cytokine-activated monocytes, as well as on mucosal sites such as the intestinal epithelium.

Direct homologs of CD1 have been found in all mammals examined to date. Although CD1 molecules, like MHC Class I molecules, are associated with β₂-microglobulin, they are structurally different. The amino acid sequence homology of CD1 proteins to class I molecules is virtually absent in the α1 domain and is very limited in the α2 domain.

Human CD1b has been shown to act as a restriction element in the presentation of several lipid and glycolipid antigens from mycobacteria to T cells. However nothing is known of the CD1 interaction with these antigens or how the antigens induce specific T cell responses. Understanding the structural interactions between CD1 proteins, T cell receptors, and lipid and glycolipid antigens could unlock the knowledge through which synthetic CD1-presented antigens could be constructed.

### SUMMARY OF THE INVENTION

This invention relates to methods of using a synthetic antigen for enhancing the immune response of mammals. The synthetic antigen is a CD1-presented antigen comprised of a hydrophobic element containing one or more branched or unbranched acyl chains which bind nonspecifically within the hydrophobic pocket of the CD1 protein and a hydrophilic element for a highly specific interaction with a T cell receptor. Methods are also provided for blocking the immune response in mammals by directly interfering with the presentation of antigens to T cells by CD1 molecules.

In another aspect, this invention relates to synthetic CD1-presented antigens for inducing or inhibiting T cell responses in mammals. The compositions can be comprised of hydrophilic and hydrophobic moieties derived from prokaryotic or eukaryotic cells, or portions can be constructed chemically. Naturally-occurring lipid or glycolipid antigens which have been altered or modified are also included as part of this invention.

### BRIEF DESCRIPTION OF THE DRAWINGS

FIGS. 1A-1D diagram the results which demonstrate identification of the antigenic glycolipid from M. phlei as glucose monomycolate (GMM).

FIGS. 2A-2D show that LDN5 recognition of GMM did not correlate with unsaturation, cyclopropanation, R group or significant chain length differences of the mycolic acid moiety.

FIGS. 3A-3B diagram the carbohydrate specific recognition of mycolyl glycolipids by LDN5.

FIG. 4 shows the structural motif for three classes of CD1b-restricted antigens.

FIG. 5 represents the interaction of the carrier lipid portion of a CD1-synthetic antigen interacting with the hydrophobic domain of a CD1 molecule.

FIGS. 6A-6B show the structure of CD1b and CD1d associated lipids (6A) and CD1c antigens (6B), mannosyl phosphodolicol (MDP) and mannosyl phosphoheptaprenol (MPP).

FIGS. 7A-7B show free alcohols which can be phosphorylated or obtained in the phosphorylated form (7A), and hexose and pentose sugars (7B) which can be coupled to phosphorylated alcohols to produce synthetic antigens.

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

### DETAILED DESCRIPTION OF THE INVENTION

This invention is based on the unexpected discovery that non-proteinaceous antigens presented to T cells by CD1 proteins have a common and purposeful structural organization which can be used to design antigens for an immune system response. More specifically, this invention provides compositions which have two common characteristics. These compounds have hydrophobic acyl domains which bind to CD1 molecules and hydrophilic domains which are recognized by and specifically bind to T cell receptors. Potential CD1-presented antigens constructed in accordance with the format provided herein can be evaluated for their ability to stimulate or inhibit T cell proliferation.

The methods of this invention encompass the use of a synthetic antigen to increase or decrease the immune response of a mammal. One method comprises inducing or enhancing a CD1-restricted T cell response by administering to a mammal a synthetic antigen comprising a single or branched acyl chain with a length of about C₁₂ to greater than C₁₀₀, which binds to a CD1 protein, and a hydrophilic moiety which is recognized by a T cell.

A synthetic antigen, in accordance with this invention, is an antigen which is not naturally occurring in an organism. That is, a synthetic antigen may be one that chemically synthesized, or parts thereof synthesized by combining elements, molecules or compounds. Further, a synthetic antigen can be constructed by combining two or more components, one or more of which is isolated or derived from an organism, thus producing a hybrid or chimeric antigen. In addition, a synthetic antigen can be an antigen which, although derived or isolated from an organism, is modified or altered to effect a different response in an organism when interacting with a CD1 protein or a T lymphocyte. For example, a synthetic antigen capable of being presented to a T cell by a human CD1⁺ APC can be constructed by isolating and combining a lipid moiety of a plant with a hydrophilic moiety which is chemically synthesized.

Thus, the components of a synthetic antigen of this invention can be selected from acyl chains which are synthesized in the laboratory or which are derived from a prokaryotic or eukaryotic organism. Acyl chains which comprise a backbone of as little as twelve carbons (C₁₂) are recognized by CD1 molecules. It is possible that shorter acyl chains can be recognized. The most effective acyl chains are those ranging in length from C₃₀ to C₉₀ whether branched or single chained. The optimal length for an acyl chain of this invention is greater than C₃₅. The hydrocarbon chains can be saturated or unsaturated.

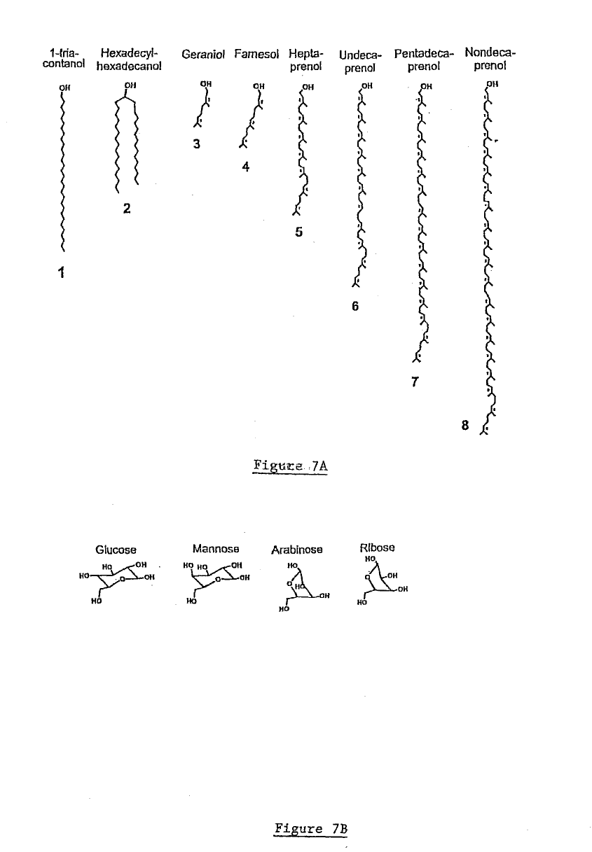

In a similar manner, the hydrophilic moiety of the antigen can be synthesized or derived from an organism. The hydrophilic portion of the antigen molecule can be a carbohydrate, such as glucose, galactose, mannose, a sugar derivative, or another hydrophilic composition. See, e.g., FIG. 7B. The carbohydrates can range from simple sugars which are ringed or linear, or they can consist of more complex structures which can include several ring structures, or sugars linked in linear or branched fashion. Preferred embodiments include carbohydrate moieties from gram-positive and gram-negative bacteria. Such carbohydrates, in addition to being derived or isolated from microbes, can be components of tumors and various cell types of eukaryotes.

The lipids and hydrophilic molecules comprising these antigens can be modified from the native state for specific purposes. For example, slight alterations in naturally-occurring carbohydrate antigens of T cells can be made which do not affect the specificity of the antigen for particular classes of T cells but which inhibit the activity of the T cell, reducing the immune response. These antigen moieties can be used as the hydrophilic portion of a synthetic antigen.

The antigens of this invention are typically prepared by standard chemical practices for the preparation of lipids, glycolipids and other lipid-like molecules. Hydrophobic and hydrophilic components can also be obtained from suppliers of such molecules (e.g., Sigma, Ribi, etc.).

Determination of Structural and Functional Characteristics

Identification of a novel CD1-restricted glycolipid antigen, glucose monomycolate (GMM), allowed a systematic analysis of the structural features that determined its recognition by T cells. Analogs of GMM that differed substantially in their acyl chain lengths and other chemical features of the lipid moiety were recognized by T cells. In contrast, T cells demonstrated fine specificity for the carbohydrate portion of mycolyl glycolipids, even discriminating among carbohydrate isomers differing only in the orientation of a single hydroxyl group. These results provide strong support for a molecular model of antigen presentation in which the acyl chains of the antigen bind relatively non-specifically within the deep, hydrophobic pocket of the CD1 protein, resulting in presentation of the hydrophilic elements of antigens for highly specific interactions with the T cell receptor (TCR). Further, this model is consistent with what is known of the structure of the CD1 molecule to date.

Human CD1 proteins are a family of non-polymorphic transmembrane glycoproteins expressed in assocation with β₂-microglobulin on the surface of antigen presenting cells (APCs) (S. A. Porcelli (1995) Adv. Immunol. 59:1-98). Unlike the well known MHC class I and class II proteins that present peptide antigens to T cells, the human CD1 proteins (CD1a, CD1b and CD1c) mediate specific T cell recognition of bacterial lipid and glycolipid antigens (S. A. Porcelli, et al. (1992) Nature 360:593-597; E. M. Beckman et al. (1994) Nature 372:691-694; Beckman, et al. (1996) J. Immunol. 157:2795-2803). Previous studies of mycobacteria specific T cells have identified two classes of CD1-restricted lipid antigens. These are the free mycolic acids, a family of α-branched, β-hydroxy long chain fatty acids, and the phosphotidylinositol containing glycolipids including lipoarabinomannan (LAM) and the phosphatidylinositol mannosides (PIMs) (Beckman, et al. (1994) supra). To identify other antigens presented by the CD1 system so that an antigen model could be defined, additional T cell lines specific for mycobacterial lipid antigens were established. Analysis of the CD4⁻ CD8⁻ TCRαβ⁺ T cell line LDN5, isolated from a cutaneous granuloma of a subject with chronic Mycobacterium leprae infection, revealed evidence for a third class of CD1 restricted lipid antigens.

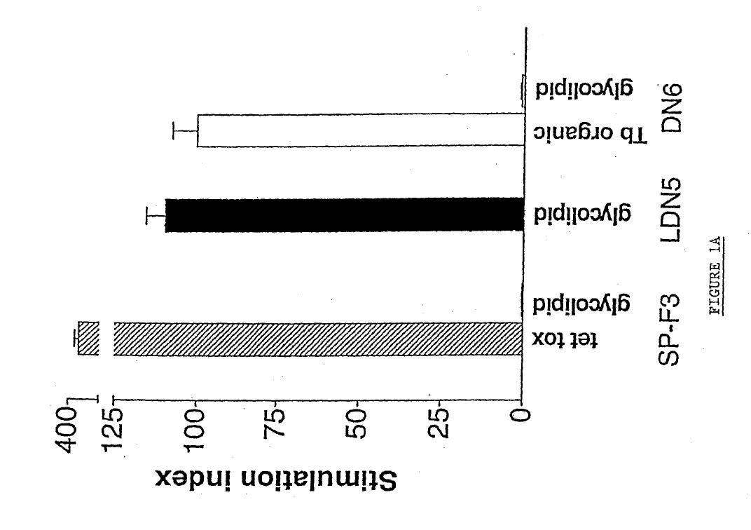

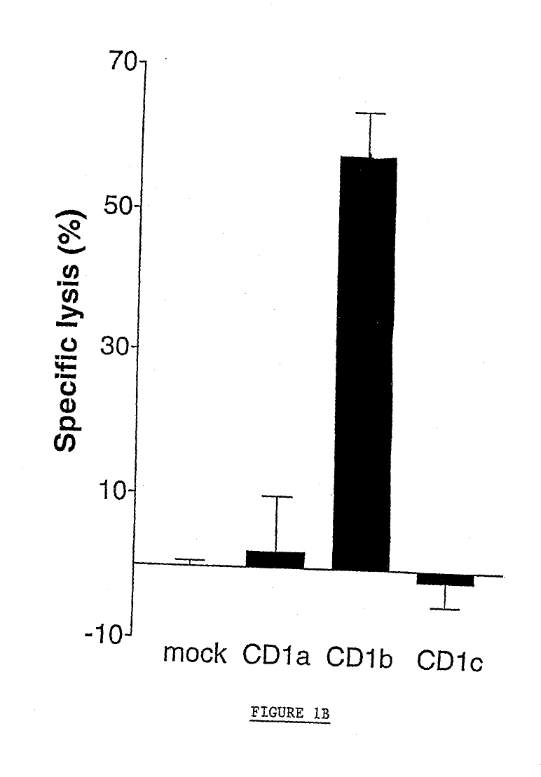

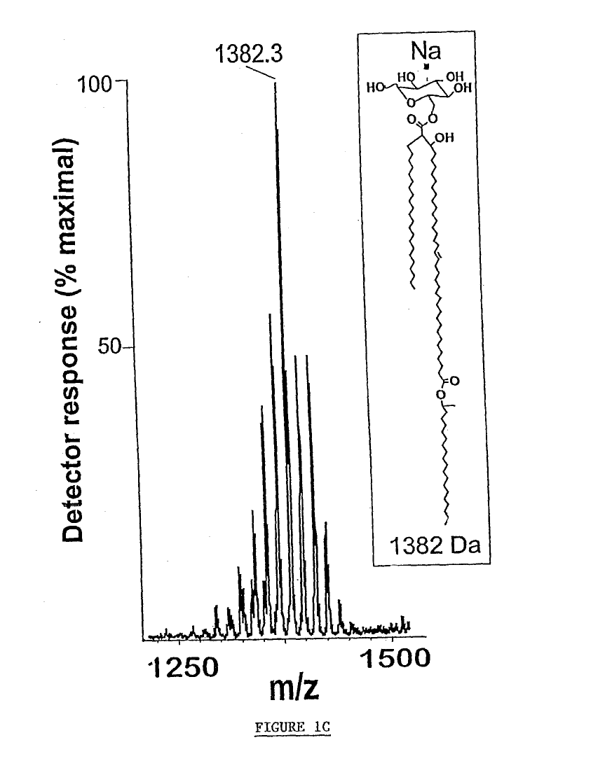

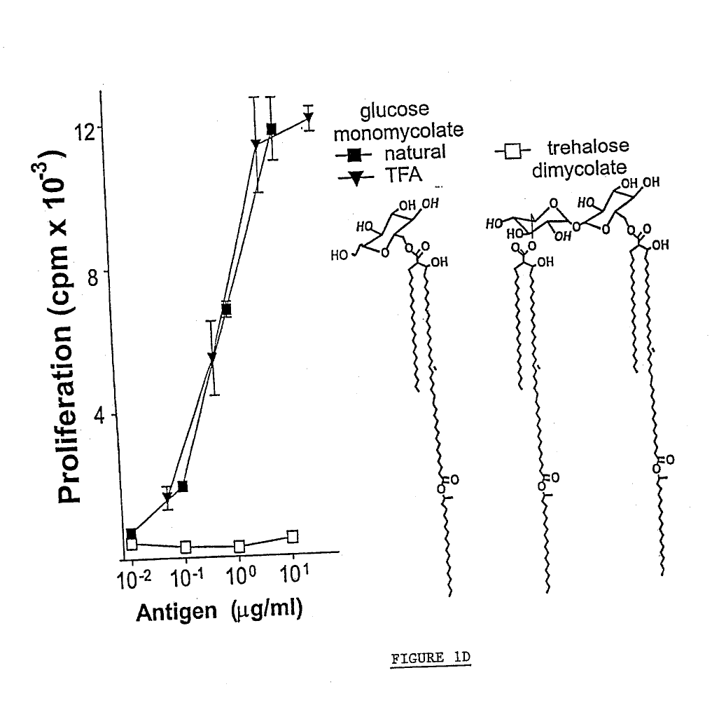

LDN5 proliferated to only one of the many lipids present in organic extracts of M. leprae separated by preparative thin layer chromatography (TLC), and cross-reacted strongly with a lipid of identical retardation factor (R_{f}=0.67) extracted from several rapidly growing mycobacterial species including M. phlei (Example 1). Analysis of the active lipid present in M. phlei organic extracts by analytical TLC revealed that the antigen contained carbohydrate, but lacked organic phosphate, distinguishing it from the two previously described classes of CD1-restricted antigens. Proliferative responses to the purified glycolipid were observed only for LDN5, but not a panel of 14 other T cell lines, including those specific for lipid (DN6) or peptide (SP-F3) antigens (M. G. Roncarolo, et al. (1988) J. Exp. Med. 168:2139), ruling out a mitogenic (non-specific) T cell stimulating activity (FIG. 1A). FIG. 1A shows that cytokine activated macrophages and the purified M. phlei antigenic glycolipid (2 mg/ml) stimulated LDN5, but not 14 other T cell lines tested, including SP-F3 (HLA-DR-restricted, tetanus toxoid specific, 10 μg/ml) and DN6 (CD1c restricted, M. tuberculosis lipid specific, 1/200 dilution), two examples shown here. The stimulation index was calculated as cpm in the presence of antigen/cpm in the absence of antigen. LDN5, DN6 and SP-F3 incorporated 123, 149 and 79 cpm, respectively, in the absence of antigen.

LDNS lysed CD1b transfected C1R lymphoblastoid target cells (Effector:Target, 25:1) cultured with 0.5 mg/ml purified antigenic glycolipid, but not similarly treated mock, CD1a or CD1c transfectants (FIG. 1B). There was no response in the absence of antigen. These results demonstrated the response of LDN5 to a novel mycobacterial glycolipid was restricted by CD1b.

The structure of the lipid and carbohydrate moieties of the antigenic glycolipid were determined separately. After alkaline hydrolysis of the antigen, products were separated into a two phase modified Folch partition from which the organic and aqueous phases were recovered. The organic phase contained lipids that coeluted on high pressure liquid chromatography (HPLC) with mycobacterial mycolic acids (E. M. Beckman, et al. (1994) Nature 372:691), and the aqueous phase showed a single product which was identified as glucose by gas chromatography (GC). This composition analysis suggested that the glycolipid antigen was glucose monomycolate (GMM), a previously described mycobacterial cell wall component consisting of a single glucopyranoside residue esterified at its sixth carbon to mycolic acid (P. J. Brennan, et al. (1969) Eur. J. Biochem. 13:117.

Electrospray ionization mass spectroscopy (ESI-MS) analysis of the intact antigenic glycolipid (FIG. 1C) revealed two superimposed alkane series of ions with the most abundant species at m/z 1382, corresponding to a Na adduct of GMM containing a monounsaturated C₈₀ wax-ester mycolic acid (G. S. Besra and D. Chatterjee in Tuberculosis, Pathogenesis, Protection and Control, Barry R. Bloom, ed. (ASM Press: Washington, D.C., 1994). ESI-MS was performed on Quattro II triple quadrupole mass spectrometer in the positive mode with samples in chloroform:methanol (2:1) at a flow rate of 2-4 μl/min. Proof that the antigen was in fact GMM was obtained by examining the ability of LDN5 T cells to respond to purified mycobacterial cord factors (α,α trehalose dimycolate) which had been treated with trifluoroacetic acid (TFA), which released GMM by cleavage at the α-glycosidic linkage. Whereas, purified cord factors from M. phlei and M. tuberculosis were not antigenic for LDN5, TFA treatment yielded GMM (“TFA” GMM) that stimulated LDN5 with a dose response that was nearly identical to the GMM (“natural” GMM) purified directly from M. phlei (FIG. 1D)

The role of the lipid structure on T cell recognition was determined by isolating GMM from mycobacterial species that differ in mycolic acid composition. M. bovis BCG, M. fortuitum, M. smegmatis and M. phlei produce GMMs consisting of glucose esterified to mycolic acids that vary with regard to acyl chain length and the presence or absence of R group substitutions, double bonds, and cyclopropane rings. M. bovis BCG and M. tuberculosis mycolic acids contain cyclopropyl groups in contrast to M. smegatis mycolic acids which contain double bonds in place of cyclopropyl groups (K. Kaneda, et al. (1988) J. Gen. Microbial. 134:2213; Y. Yuan, et al. (1995) Proc. Nat. Acad. Sci. USA 92:6630). These species differ in expression of mycolic acids either containing no R groups (i.e., α and α′ mycolates) or containing keto, methoxy, epoxy or wax-ester R groups as follows: M. tuberculosis (α, keto, methoxy); BCG (α, keto); M. phlei (α, wax-ester and possibly small amounts of keto) M. fortuitum and M. smegmatis (α, α′, epoxy) (D. E. Minnikin, et al. (1984) Arch. Microbio. 139:225; R. E. Lee, et al. (1996) Curr. Top. Microbiol. and Immunol. 215:1).

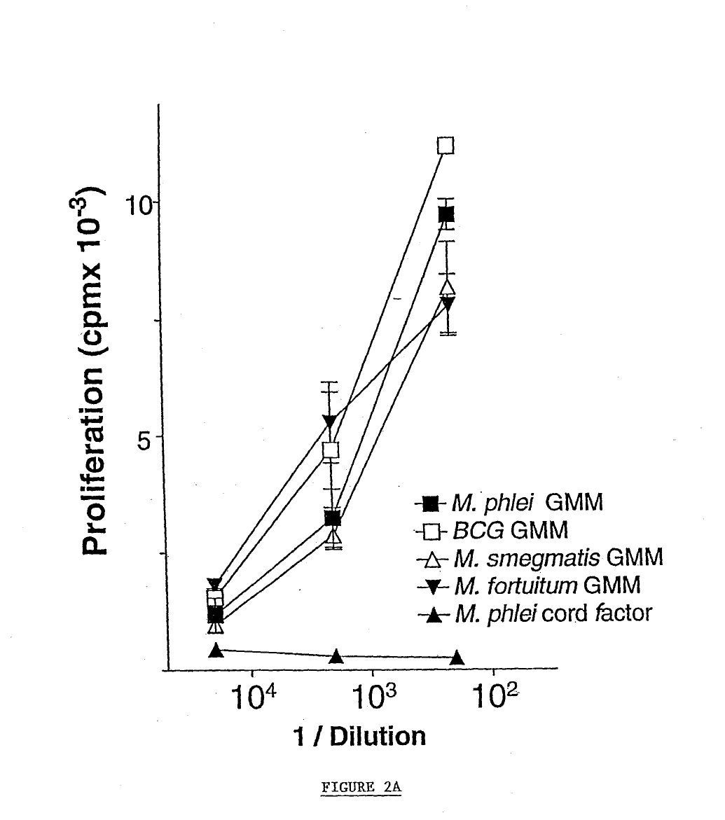

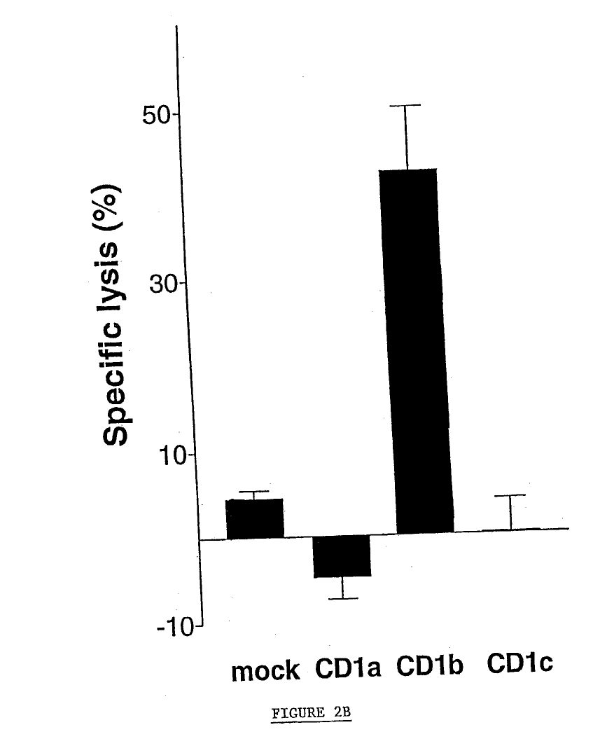

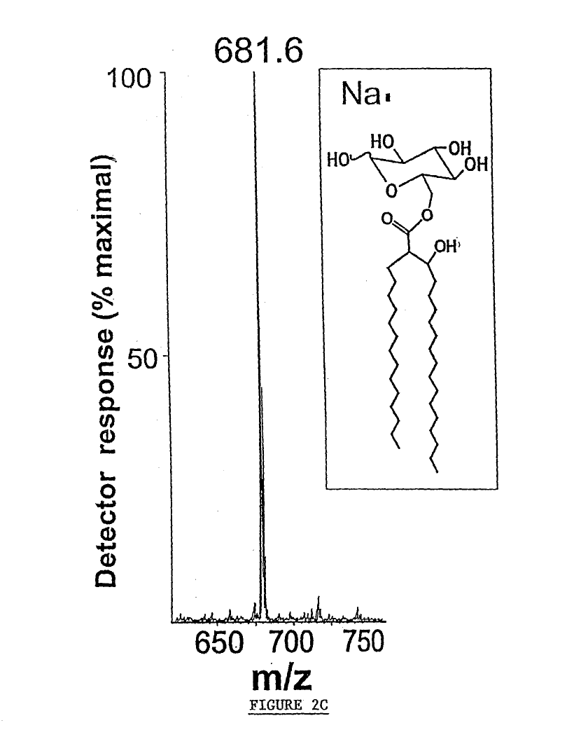

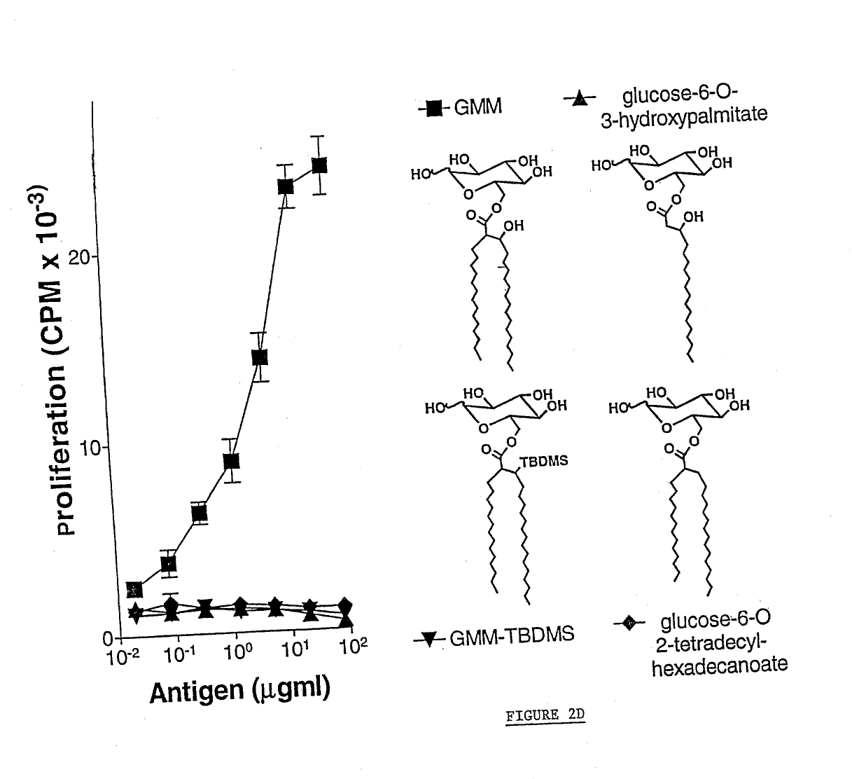

LDN5 responded to each of these different GMMs at equivalent doses, indicating that the naturally-occurring structural variations of the hydrophobic tails of the antigen were unlikely to determine specific T cell responses (FIG. 2A). This result was definitively confirmed by the CD1b-restricted response of LDN5 to a fully synthetic GMM containing a C₃₂ mycolic acid (FIGS. 2B and 2C). LDN5 lysed C1R lymphoblastoid target cells (E:T, 25:1) transfected with CD1b and cocultured with synthetic GMM (5 μg/ml). Similarly treated mock, CD1a or CD1c transfected cells were not lysed (FIG. 2B). The Na adduct of the fully synthetic GMM containing C₃₂ mycolic acid was detected by ESI-MS analysis to be a single ion peak at m/z 681.6 (FIG. 2C). This synthetic GMM antigen lacks long chain length (compared with C₈₀ mycolic acids of mycobacteria), cyclopropanation, double bonds and R groups, ruling out all of these natural chemical variations of the mycolic acid moiety as necessary antigenic determinants. The discovery of a synthetic CD1-restricted antigen with a simple and well defined structure allowed the systematic study of individual molecular features of the antigen that determined the specificity of the T cell response.

Since long chain length and naturally occurring chemical substitutions of the mycolic acid were not crucial for presentation and recognition of the antigen, it was determined whether the spectrum of glycolipids recognized by LDN5 were extremely broad (i.e., any glucosylated lipid) or was limited to mycolyl glycolipids. Mycolyl glycolipids are defined by the α-branched, β-hydroxy structure of the mycolic acid, so analogs of GMM lacking these defining features were synthesized to test their role in T cell recognition. Previously described methods for mycolic acid synthesis, TBDMS derivatization and hexose-6-O-acyl preparation (A. K. Datta, et al. (1991) Carbohydrate Research 218:95) were followed except that the appropriate lipid, 3-hydroxypalmitate (Matreya), tetradecylhexadecanoate (Wako), triacontanoate (Sigma), or the appropriate carbohydrate, glucose, mannose, galactose (Sigma), were substituted in the reactions. “Natural” hexose mycolates were isolated from M. phlei grown in glucose, galactose or mannose supplemented media (Y. Natsuhara, et al. (1990) Cancer Immunol. Immunother. 31:99). All lipid structures were confirmed by ESI-MS and TLC. Nuclear magnetic resonance analysis of semi-synthetic hexose monomycolates (Bruker ACE-300) revealed a low field chemical shift of H-6ₐ (δ 4.51, doublet) and H-6_{b} (δ 4.06, double doublet) indicative of acylation at the position 6 hydroxyl.

LDN5 did not respond to glucose 6-O-3-hydroxypalmitate, a glycolipid identical to GMM except for its lack of the α carbon branch. Likewise, removal or derivatization of the β-hydroxyl of the mycolic acid abolished the T cell response entirely (FIG. 2D). In addition, LDN5 did not respond to a variety of non-mycolyl glycolipids that were similar in structure to GMM, containing glucose linked to acyl chains of approximately C₃₂. Therefore, recognition of the GMM was absolutely dependent on the α-branched, β-hydroxy lipid structure that defines mycolyl lipids, but long distally substituted acyl chains (branchs) were not required.

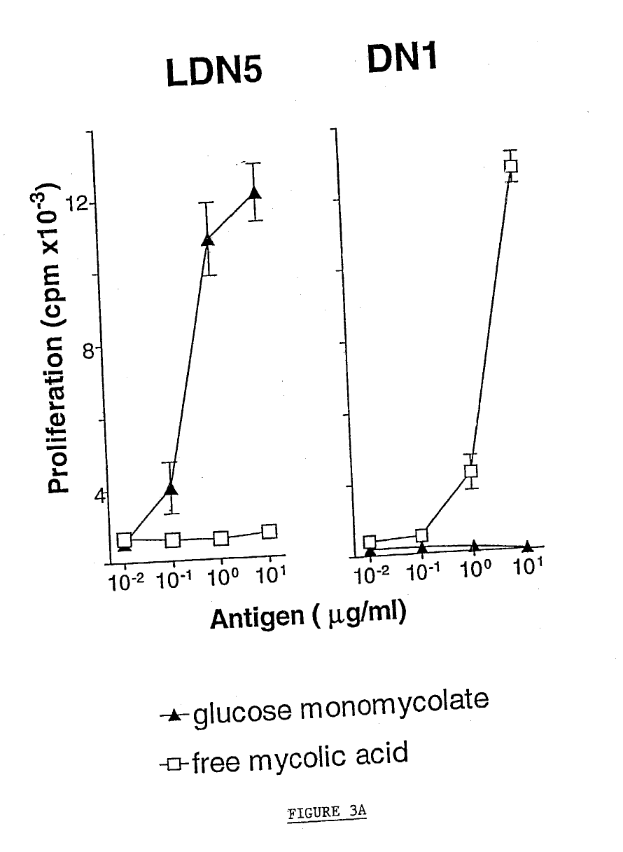

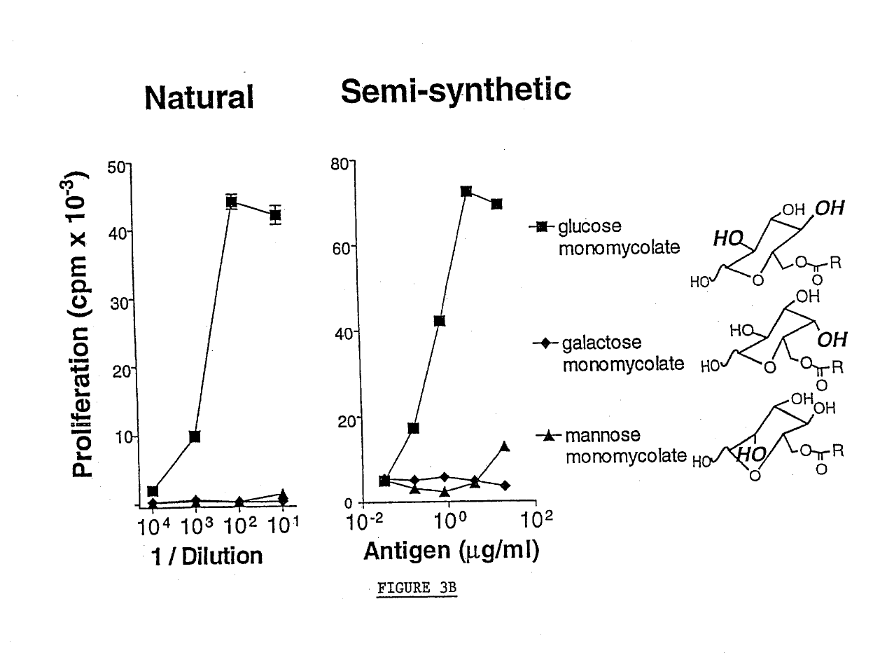

The role of the carbohydrate moiety of the glycolipid in T cell recognition was separately evaluated. The CD1b-restricted response of LDN5 to GMM was carbohydrate dependent, since intact free mycolic acids were not antigenic for LDN5 (FIG. 3A). Although the carbohydrate moiety of GMM could in theory have contributed to antigenicity by facilitating APC uptake or processing of the antigen, analysis using the CD1b-restricted T cell line DN1, which is specific for free mycolic acid, revealed that this was unlikely. DN1 responded to free mycolic acid and not to GMM, whereas LDN5 showed the opposite pattern of recognition of these two antigens (FIG. 3A). This result indicated that activated macrophages did not chemically interconvert these antigens, and were capable of taking up, processing and presenting both antigens in a CD1b-restricted manner. Thus, the carbohydrate dependence of GMM recognition by LDN5 was a specific feature of this CD1b restricted T cell line and suggested that the glucose component of the antigen was directly involved in T cell recognition of this glycolipid.

To investigate the specificity of the T cell response for the carbohydrate moiety of the antigen, a variety of differentially glycosylated mycolic acids were purified. The structure of the carbohydrate was crucial for the T cell response, as LDN5 responded to M. tuberculosis GMM but not several M. tuberculosis mycolyl lipids containing carbohydrates other than glucose such as glycerol mycolate, trehalose monomycolate and arabinomycolate. To examine the T cell response to mycolyl glycolipids most similar to GMM, two stereoisomers of GMM, mannose monomycolate and galactose monomycolate, were prepared (A. K. Datta, supra; Y. Natsuhara, supra). LDN5 proliferated at similar doses to natural and semi-synthetic GMM. In contrast, LDN5 responded very weakly or not at all to mannose monomycolate and galactose monomycolate, stereoisomers that differ from GMM only by the orientation of the hydroxyl group at the 2 or 4 positions of the pyranose ring, respectively (FIG. 3B). Thus, these T cells showed extraordinary fine specificity for the carbohydrate moiety of GMM, discriminating among stereoisomers varying in structure only in the orientation of a hydroxyl group on the pyranose ring.

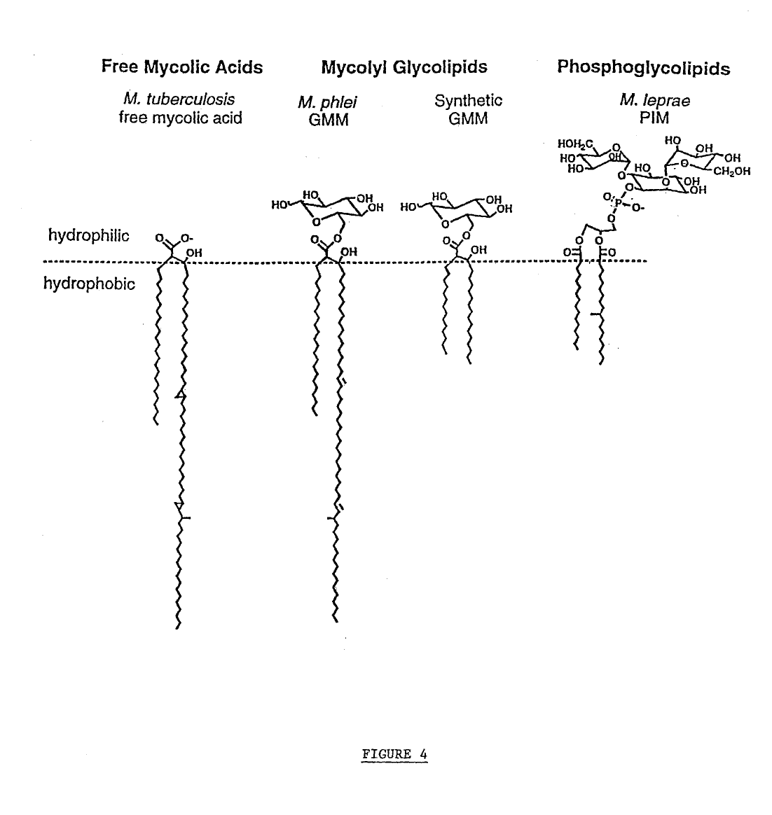

The identification of GMM as a CD1-restricted antigen and the analysis of its structural features that determined T cell recognition revealed a general motif for CD1-restricted glycolipids as divergent in structure as mycobacterial phosphoglycolipids and free mycolic acids (FIG. 4). The CD1-restricted recognition of a synthetic GMM proved that the long hydrophobic tails of the antigen can be stripped of all chemical substitutions and shortened from approximately C₇₀ to C₃₂ and still retain its ability to stimulate T cells (FIG. 2B), as long as the α-branched, β-hydroxy structure of the lipid was maintained (FIG. 2D). Notably, the proximally branched mycolic acid of this synthetic antigen had an overall acyl chain length similar to that of the combined acyl chains of PIM or LAM. Thus, CD1-restricted antigens from each of the three classes share a general structure in which a single proximally branched acyl chain (free mycolic acid, GMM) or two acyl chains (LAM, PIM) comprised of 32 or more carbon atoms are capped with a hydrophilic moiety (FIG. 4). Subtle changes in the structure of the hydrophilic cap of GMM, such as removal of the β-hydroxyl of the mycolic acid (FIG. 2D) or a change in the orientation of a hydroxyl group in the carbohydrate moiety (FIG. 3B), abolished T recognition entirely. Thus, the T cell response was highly specific for the structure of the hydrophilic cap, but not the fine structure of the hydrophobic acyl chains of GMM. These findings significantly extend previous studies showing that large changes in the hydrophilic caps of LAM or mycolic acid alter T cell recognition (E. M. Beckman (1994) supra; P. A. Sieling (1995) supra).

Further analysis of CD1b-restricted T cell responses to GMM showed that the binding of GMM to CD1b was pH dependent, occurring at pH 4.0 but not at pH 7.0. A synthetic GMM (sGMM) that contained a shorter branched acyl chain also bound at pH 4.0 but not at pH 7.0. sGMM bound to CD1b but not to chips coated with HLA-A2 (human leukocyte antigen-A2) or HLA-DR1, which served as negative controls.

Glucose-6-o-triacontanoate (G6T), which differs from GMM by possessing a single unbranched acyl chain but has almost the same number of aliphatic carbons-as sGMM, did not bind to CD1b. Thus, CD1b binds to both natural and synthetic GMM but not to an analog that has only one long alkyl chain instead of two short alkyl chains.

The uptake and processing pathway for CD1b-presented antigens was also defined. Several steps in the presentation of two related classes of CD1b-presented antigens, free and glycosylated mycolates, were examined. T cell recognition of GMM was blocked by agents that fix APC membranes or neutralized the pH of endosomes, indicating a requirement for GMM uptake into an acidic compartment prior to recognition. Different T cell lines responded to free mycolate or GMM without cross reactivity, yet both antigens were taken up by APCs at the same rate, thus demonstrating that differential recognition of these antigens resulted from T cell specificity for their hydrophilic caps and that APCs were unable to interconvert these antigens by enzymatic or chemical deglycosylation or glycosylation. APCs were also unable to cleave mycobacterial trehalose dimycolate (TDM) at its most chemically labile linkages to yield antigenic free mycolates or GMM. These results indicate that these mycolate-containing antigens are resistant to chemical or enzymatic cleavage by APCs, suggesting that molecular trimming is not a universal feature of lipid antigen processing. Thus, larger lipids, such as trehalose dimycolate cannot be broken down into GMM within cells. Given that the hydrophobic groove can accommodate approximately 32 CH₂ units when measured in crystalline form, it is likely that the CD1 protein can open to encompass larger alkyl chains or that the chains can protrude from the groove.

The potency of T cell recognition of natural and synthetic analogs of GMM containing mycolic acids between about 80-12 (C₈₀ to C₁₂) was examined to define the role of chain length. Natural GMMs were purified from M. phlei (C₇₇₋₈₃), N. farcinica (C₃₃₋₄₁), and R. equi (C₂₉₋₃₅). Additionally, fatty acids of various chain lengths were condensed to yield synthetic C₃₂, C₂₈, C₁₆, and C₁₂ mycolates that were subsequently esterified to glucose at the 6 position to make GMM. The GMM analogs were presented by CD1b. The potency of GMM analogs varied directly with the chain length across the spectrum of lipid size, with the shorter chains having less antigenic potency.

CD1d-presented glycolipids also conform to the motif for CD1b-presented antigens. Gylcosyl phosphatidylinositols and glycosyl ceramides, amphipathic glycolipids which have two alkyl chains and a hydrophilic head groups, are presented by CD1d in both humans and mice. Spada, F. M., et al. (1998) J. Exp. Med., in press.

The identification of this motif provide the molecular structure and characteristics through which new foreign and potentially self lipid antigens can be identified. In fact, these results prove that glycolipids with short chain mycolic acids characteristic of no n-mycobacterial actinomycetes such as Corynebacterium diptheriae and Nocardia asteroides can be presented by CD1 proteins, extending the range of human pathogens harboring antigens presented by the CD1 system.

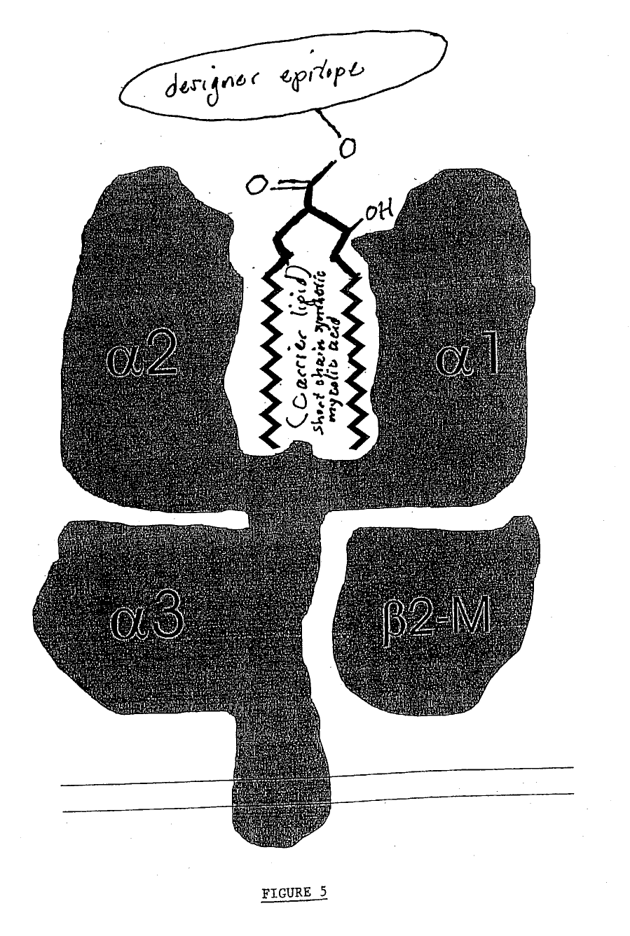

These results also reveal a molecular model for lipid antigen presentation by CD1 proteins. The carbohydrate specific recognition of mycolyl glycolipids occurs as a result of relatively non-specific hydrophobic interactions between the acyl chains of the antigen and the binding groove of CD1, leading to presentation of the hydrophilic cap of the antigen for highly specific interactions with the T cell receptor (TCR). Z-H. Zeng, et al. ((1997) Science 277:339-345) have described the crystal structure of a murine CD1 protein in which the α1 and α2 domains form a deep, bifurcated hydrophobic antigen binding pocket that is sequestered from aqueous solvent except through a narrow portal lined with polar and charged amino acids. Based on the size, shape and electrostatic topography of the CD1 antigen binding pocket, as well the structures of other known lipid binding proteins, the CD1 ligand binding groove is the likely site to interact with lipids conforming to the CD1 antigen motif with the acyl chains buried deeply within the groove, binding in the hydrophobic pockets (FIGS. 4 and 5). The polar or charged elements of the ligand can interact with amino acids near the portal. (FIG. 5). In the case of GMM presentation by CD1b, this structural model places the mycolic acid β-hydroxy group, carboxylate ester and pyranose ring of the glycolipid antigen near the portal of the alpha-helical face of CD1 which is predicted to interact with the TCRs of CD1-restricted T cells. Thus, a novel molecular model is provided, wherein the CD1 protein binds the hydrophobic portion of the amphipathic lipid resulting in presentation of polar or charged antigenic determinants to the TCR.

Additional studies involving CD1c antigen presentation substantiate this model and provide further characteristics of a CD1-presented antigen. This work also defines the structure of the first antigen presented by CD1c. The antigen is mannosyl phosphodolichol (MPD), a member of a class of long chain isoprenoid lipids that are present in all cellular organisms.

Preliminary studies indicated that the TCR and CD1c mediate human T cell responses to semi-synthetic analogs of both foreign (mycobacterial) and self (human) MPD, thus defining the first potential lipid autoantigen for αβ T cells. A trimolecular model of this recognition predicts that CD1 presents amphipathic glycolipids by sequestering the lipid within the hydrophobic groove of CD1, resulting in presentation of the carbohydrate moiety of the antigen to the TCR. Because it is the first defined glycolipid autoantigen, it determines new uses for CD1 glycolipid technology.

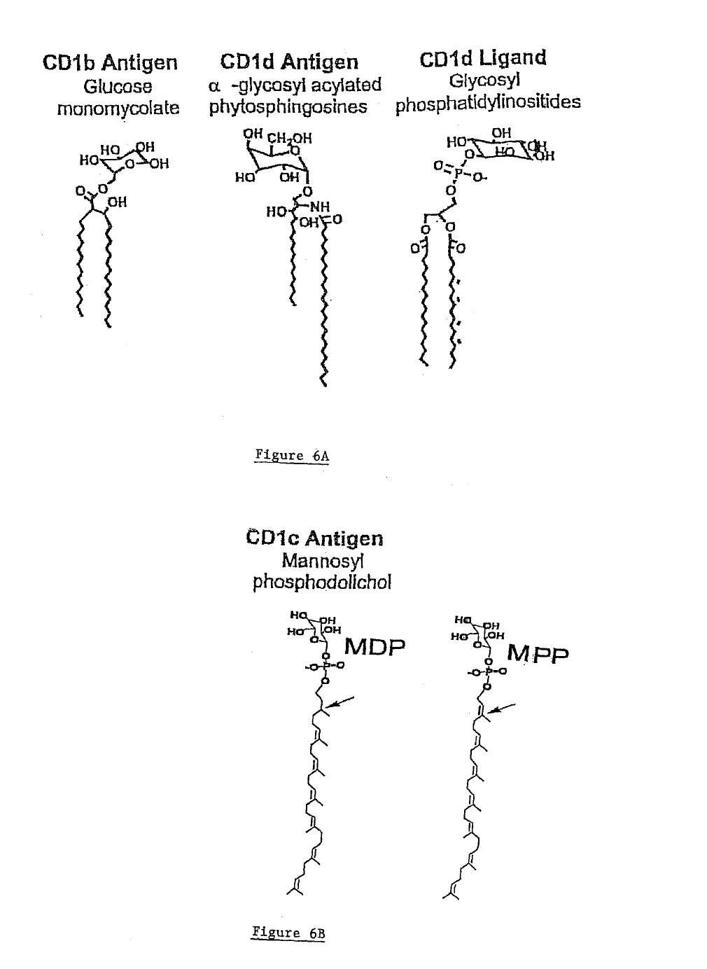

In contrast to all other known CD1-presented antigens, this glycolipid has only one lipid chain instead of two (FIG. 6B). The structure of CD1c-presented antigens was unknown, so silica chromatography was used to purify the mycobacterial lipid presented to the human CD1c-restricted αβ T cell line CD8-1 (Beckman, et al. (1996) J. Immunol. 157:2795-2803). CD8-1 was derived using M. tuberculosis antigen, but recognized lipids from a rapid-growing strain of M. avium. An anionic lipid from M. avium with a mass to charge ratio (m/z) of 679.6 was found to stimulate CD8-1. Recognition of this lipid was highly specific, as, as CD8-1 did not respond to any mycobacterial fraction that did not contain an ion of m/z 679.6. Pretreatment of APCs with mAb against CD1c locked the proliferative response of CD8-1. Thus, the T cell recognition of this lipid was specific and restricted by an antigen-presenting molecule, two features of TCR-mediated antigen recognition. To directly demonstrate the role of the TCR in recognition, the TCR α and β chains of CD8-1 were cloned and transfected into a TCR⁻ T lyphoblastoid cell line J.RT3. Transfection of the TCR chains conferred upon the recipient line the ability to specifically recognize this antigen in a CD1c-restricted manner, proving that the TCR mediates the T cell response to this glycolipid.

A partial structure for the M. avium lipid was determined by tandem ESI-MS revealing a fragmentation pattern characteristic of a hexose phospholipid with a lipid component of m/z 420. Gas chromatography (GC) of acetylated hydrolysis products revealed the hexose to be a mixture of mannose and glucose. Based on a survey of known mycobacterial phospholipids, the leading candidate structure for this antigen was that of a glycosyl phosphopolyprenol (GPP). See, Besra, G. S., et al. (1994) Proc. Natl. Acad. Sci. USA 91:12735-12739. The fragmentation pattern was consistent with the identification of the antigenic M. avium as a GPP, presuming that the lipid of m/z 420 was the hexaprenyl component of a fully saturated hexose phosphohexaprenol. To assess this, the ability of the T cell line CD8-1 to recognize purified mycobacterial GPPs was tested. The structure of Myc-PL, a mannosyl phosphopolyprenol esterified to a mycolic acid, had been determined in detail. Besra, G. S., et al. (1994) supra. Pure myc-PL was cleaved with strong base to yield free mycolate and mannosyl phosphoheptaprenol (MPP). Besra, G. S., et al. (1994) supra. CD8-1 did not recognize the intact mycolated lipid or the free mycolic acid released after acid treatment, but did respond to the pure MPP. This result was further confirmed by the response of CD8-1 to semi-synthetic GPPs made in a cell free system. Rush, J. S., et al.(1993) J. Biol. Chem. 268:13110-13117. CD8-1 responded significantly to semi-synthetic MPP [β-D-mannopyranosyl-1-monophosphoryl-(poly-cis, di-trans, α-unsaturated) heptaprenol], although the magnitude of this response was comparatively weak. More strikingly, these same T cells recognized the 2,3 dihydro analog, mannosyl phosphodolichol (MPD), at much lower concentration, and gave responses to this compound that were comparable in strength to those stimulated by native mycobacterial antigens. Moreover, two other CD1c-restricted T cell lines derived from different donors recognized MPD, suggesting the MPD could be a dominant antigen recognized by CD1c-restricted T cells specific for mycobacteria. MPDs are not nonspecific T cell mitogens, as they did not stimulate a variety of CD1a, CD1b and MHC class I restricted T cell lines. Thus, these studies established that CD1c-restricted T cells specifically recognized GPPs from four different sources, including both natural and synthetic analogs of this structure.

Long chain polyprenol compounds are ubiquitously found in all living organisms. They play essential roles in protein glycosylation in eukaryotes and in certain bacteria, and are required for cell wall synthesis by prokaryotes. These functions relate to the ability of these compounds to facilitate the translocation of carbohydrates across biological membranes and to act as sugar donors. The fine chemical details of the isoprenoid glycolipids differ systematically among different phyla of organisms. Rip, J. W. (1985) Progress in Lipid Research 24:269-309. For example, archebacteria and eukaryotes synthesize polyisoprenoids in which the most proximal isoprene unit in the chain (α-isoprene unit) is saturated. These α-saturated polyisoprenoids are generally referred to as dolichols. In contrast, most prokaryotes make α-unsaturated polyisoprenoid polyprenols. Among dolichol producing organisms, there are differences in the length of the isoprenoid lipids (FIG. 7A), with those produced by archebacteria being relatively short (C₃₅) whereas those found in protozoa, fungi and mammals being much longer (C₅₀₋₆₅, C₇₅₋₈₅ C₉₀₋₁₀₀, respectively). Krag, S. S. (1998) Biochem. Biophys. Res. Comm. 243:1-5; Hemming, F. W. (1992) Biochem. & Cell. Biol. 70:377-381. The degree of phosphorylation and identity of the carbohydrates of GPPs are also typical of certain classes of organisms. For example, glucosyl and mannosyl phosphodolichols are present in higher eukaryotes, whereas mycobacteria synthesize arabinosyl and ribosyl analogs as well. Thus, it appears that GPPs represent a widespread class of ubiquitously distributed glycolipid antigens which the mammalian immune system evolved to recognize as a prominent and distinctive component of many pathogens. This then provides a new class of antigens which can be presented by CD1 proteins and a wider range of organisms involved in the CD1-restricted immune activities of mammals. Further, this group of glycolipids comprises phosphorylated prenols and phosphoprenols some of which have been shown to improve host response to viral infections in mammals.

All three CD1-restricted lines preferentially recognized the α-saturated MPDs typical of eukaryotes more potently than the α-unsaturated GPPs typical of prokaryotes. Krag, S. S. (1998) Biochem. Biophys. Res. Comm. 243:1-5. To determine if human T cells would recognized a human MPD structure, β-D-mannopyranosyl-1-monophosphoryl-(poly-cis, di-trans, α-saturated) nondecaprenol was synthesized. Human T cells (line CD8-1) responded significantly to this compound which conforms to the structure of a self lipid, thus defining the first lipid autoantigen for αβ T cells. Significantly, T cells which respond directly to CD1c-expressing cells in vitro in the absence of any apparent added antigen (CD1c-autoreactive T cells) have been repeatedly isolated from normal human donors. Porcelli, S., et al. (1989) Nature 341:447-450. Given the novel discovery that CD1c presents a self GPP, this autoreactivity could have been directed at CD1c proteins which contained bound endogenous cellular GPPs. In fact, three of four CD1c-autoreactive T cell lines, but not CD1a-autoreactive or CD1b-restricted T cell lines in the present study showed augmentation of their responses to CD1-expressing cells in the presence of the exogenous hexose phospholipid (most likely a GPP) purified from M. avium. For one of these T cell lines (JR-1), reduction of the number of CD1c⁺ APCs in the culture caused loss of the autoreactivity, whereas the response to the exogenous hexose phospholipid was still strongly detected. This indicates that the responses of many autoreactive CD1c-specific T cells could actually be modulated by the levels of endogenous self GPPs, which could have significant implications for mechanisms which lead to autoimmunity.

The structures elucidated herein provide the chemical parameters by which synthetic antigens can be constructed and used as immunomodulators in the treatment of infectious and autoimmune diseases, and in tumor suppression. For example, a method for inducing a CD1-restricted T cell response can comprise administering to a mammal a synthetic antigen comprising one or more branched or unbranched acyl chains which bind to a CD1 protein and a hydrophilic moiety which is recognized by a T cell. Further, methods for treating a disease in vertebrate animals, especially mammals, can comprise administering to the vertebrate a synthetic composition which induces a CD1-restricted immune response. The immune response comprises T cell recognition of a hydrophilic component of the composition associated with the disease, wherein the hydrophilic component is conjugated to a hydrophobic component which comprises one or more saturated or unsaturated acyl chains. The acyl chains associate with the hydrophobic groove of a CD1 molecule on an antigen-presenting cell. A synthetic antigen can even comprise one branched acyl chain consisting of a free mycolate which is recognized by a T cell. The acyl chain of this antigen can be covalently bonded to a phosphate group (PO₄⁻) wherein the β and γ carbons of the acyl chain are saturated. Alternatively, the β and γ carbons of the acyl chain are unsaturated.

It is expected that the acyl chains of the hydrophobic moiety will range in length from about C₁₂ to greater than C₁₀₀ and can be saturated or unsaturated chains; however, it is possible that longer chains will work and that there could be further processing of glycolipid antigens by APCs. Most likely, one or more of the acyl chains of an effective synthetic antigen will have length of C₃₀ to C₉₀. Given that many types of natural CD1-presented antigens are phospholipids, it is expected that many synthetic antigens used in the methods of this invention will comprise phospholipids, wherein one or more of the acyl chains is covalently bound to a phosphate group (PO₄⁻).

The hydrophilic moiety which is specifically recognized by the TCR can be any hydrophilic substance: polypeptide, carbohydrate, smaller hydrophilic molecules and the like. Preferably, the hydrophilic cap is a carbohydrate, such as a glucose or mannose unit. The hydrophilic moiety to which the T cell response is induced can be derived from or isolated from a viral, bacterial, fungal, parasitic, tumor, or auto antigen.

Further, these synthetic antigens can be administered with an adjuvant, a peptide, and/or an additional antigen, such as an MHC class I or MHC class II antigen to enhance the immunogenic response(s).

The synthetic antigens of this inventions can also act as immunoregulatory agents, downregulating or upregulating an immune response through activation of T cells which can, for example, downregulate a response to another antigen. Thus, methods are provided for modulating immune responses which are not CD1-restricted by inducing a CD1-restricted T cell response to a synthetic antigen.

The CD1-presented antigens of the present invention can be administered to vertebrate animals, including mammals. The vaccines of this invention will have both human and veterinary applications as prophylactic and therapeutic vaccines. Further, when used therapeutically, these vaccines can be combined with chemotherapy to produce a more effective treatment of many diseases. CD1-presented synthetic antigens can also be combined with other antigens, either another CD1-presented antigen or an MHC Class I or MHC Class II-presented antigen, to produce a more effective prophylactic or therapeutic vaccine.

The antigens of this invention can be employed in admixture with conventional excipients; i.e., pharmaceutically acceptable organic or inorganic carriers which do not deleteriously react with the immunologically-active components and which are suitable for parenteral, mucosal, or even topical applications. Such a carrier includes but is not limited to saline, buffered saline, dextrose, water, glycerol, ethanol, oils, and combinations thereof. The carrier and composition can be sterile. The formulation should suit the mode of administration. Parenteral administration can include the introduction of substances into an organism by intravenous, subcutaneous or intramuscular means, including by implant. Mucosal administration includes pulmonary, intranasal, oral, vaginal, or rectal administration.

The carrier can be added to the vaccine at any convenient time. In the case of a lyophilized vaccine, the carrier can, for example, be added immediately prior to administration. Alternatively, the final product can be manufactured with the carrier.

The present invention provides a variety of pharmaceutical compositions. Such compositions comprise a therapeutically (or prophylactically) effective amount of a synthetic antigen or a CD1:antigen complex, and a carrier. The composition, if desired, can also contain minor amounts of wetting or emulsifying agents, or pH buffering agents, or preservatives. Typical preservatives can include, potassium sorbate, sodium metabisulfite, methyl paraben, propyl paraben, thimerosal, etc.

The composition can be a liquid solution, suspension, emulsion, tablet, pill, capsule, sustained release formulation, or powder. The method of administration can dictate how the composition will be formulated. For example, the composition can be formulated as a suppository, with traditional binders and carriers such as triglycerides. Oral formulation can include plant materials, T. A. Haq, et al., (1995) Science 268:714-716, or standard carriers such as pharmaceutical grades of mannitol, lactose, starch, magnesium stearate, sodium saccharine, cellulose, magnesium carbonate, etc.

In a preferred embodiment, the synthetic antigens are administered without an adjuvant. A variety of adjuvants can also be used to amplify cell-mediated and humoral responses when mixed with a CD1-presented synthetic antigen. The adjuvant of choice for human administration is an aluminum salt such as alum, aluminum hydroxide or aluminum phosphate. Other adjuvants, for example, oil-based emulsions that contain biodegradable materials, can be tested in combination with the antigen and found to be effective and safe. Adjuvants that are oil-based emulsions include Syntex formulation SAF-1, Ciba-Geigy formulations, and Ribi formulation. See, N. R. Rabinovich et al. (1994) Science 265:1401-1404. Freund's incomplete or complete adjuvants can also be effective.

Methods of administration will vary in accordance with the type of disorder and microorganism sought to be controlled or eradicated. The dosage of the vaccine will be dependent upon the amount of antigen, it's level of antigenicity, and the route of administration. A person of ordinary skill in the art can easily and readily titrate the dosage for an immunogenic response for each antigen and method of administration.

For parenteral application, particularly suitable are injectable, sterile solutions, preferably oily or aqueous solutions, as well as suspensions, emulsions, or implants, including suppositories. For enteral or mucosal application (including via oral and nasal mucosa), particularly suitable are tablets, liquids, drops, suppositories or capsules. A syrup, elixir or the like can be used wherein a sweetened vehicle is employed. Topical application can also be used for example, in intraocular administration. Alternative methods of administration can include an immune-stimulating complex (ISCOM) as described in U.S. Pat. No. 4,900,549 (or European Patent Publication No. 0 604 727 A1 (Publ. Jul. 6, 1994). In addition, viral vectors, liposome and microspheres, and microcapsules are available and can be used. See, Rabinovich, supra.

For diseases of the lungs, such as tuberculosis, pulmonary administration may be preferred for prophylactic purposes or for immediate and specific localized treatment. Pulmonary administration can be accomplished, for example, using any of various delivery devices know in the art. See, e.g., S. P. Newman (1984) in Aerosols and the Lung, Clarke and Davia (eds.), Butterworths, London, England, pp. 197-224; PCT Publication No. WO 92/16192; PCT Publication No. WO 91/08760; NTIS Patent Application 7,504,047 (1990), including but not limited to nebulizers, metered dose inhalers, and powder inhalers. Various delivery devices are commercially available and can be employed, e.g., Ultravent nebulizer (Mallinckrodt, Inc., St. Louis, Mo.); Acorn II nebulizer (Marquest Medical Products, Englewood, Colo.). Such devices typically entail the use of formulations suitable for dispersing from such a device, in which a propellant material may be present.

The discovery of a structural motif for antigen presentation by CD1 proteins provides the means by which synthetic antigens can be used to extend the spectrum of antigens presented by CD1 molecules and provides the opportunity for vaccines comprising CD1-presented synthetic antigens that are effective against all gram negative and most gram positive bacteria (including Streptococcus sp. and Staphylococcus sp.), and a variety of parasitic protozoa. All gram negative bacteria contain lipopolysaccharides (LPS) which are similar in structure to lipomannans. Most gram positive bacteria contain structurally-related glycolipids such as lipoteichoic acids. In addition, the chemical composition of many disease-causing protozoa includes glycolipids such as the lipophosphoglycans of Leishmania. Orlandi, P. A. and S. J. Turco, J. Biol. Chem. 262:10384-10391. It is likely that the cell walls and other cellular components of the fungi also contain lipoglycans; therefore, vaccines comprising CD1-presented synthetic antigens can be used to prevent or treat fungal infections of vertebrates. These antigens can include partial derivatives of mycolic acid, LAM, GMM, PIM, MPP, or MPD molecules or derivatives of similar glycolipids from microbial organisms, whether prokaryotic or eukaryotic in nature.

At present, vaccines against protozoan parasites are either nonexistent or not feasible for mass immunization. See, e.g., Nussenzweig, R. S. and C. A. Long (1994) Science 265:1381-1383. Examples of diseases caused by protozoa include, but are not limited to, malaria, trichinosis, filariasis, trypanosomiasis, schistosomiasis, toxoplasmosis and leishmaniasis. Protozoan infections can be more difficult to control and eradicate than bacterial infections because compounds that kill a protozoan parasite are often toxic to the host. For example, most of the drugs used to treat diseases caused by Trypanosoma species can cause serious side effects and even death. In addition, drug resistance of many protozoal species is becoming increasingly common in most parts of the world.

The possibility of synthetic antigens provided by this invention opens the door to a whole new class of vaccines based on the T-cell proliferation to a CD1-presented antigens. Vaccines incorporating synthetic antigens either by themselves or combined with a protein antigen could prove an efficacious and cost-effective treatment against protozoan parasites. An advantage of such a vaccine is that toxic medicaments may not have to be administered or can be administered in reduced dosages in conjunction with a CD1-presented synthetic antigen to control the infection.

Further, synthetic antigens can be made very pure, while antigens isolated from microbial organisms are frequently contaminated with other proteins which can be included in a final product such as a vaccine. These extraneous contaminants can cause undesirable side reactions in a mammal such as a human. Synthetic antigens can be synthesized and purified and used without the risk of undesirable contaminants.

The synthetic antigens provided by this invention can also be used to prevent or reduce autoimmune responses in reactions to foreign antigens or in autoimmune diseases such as Graft vs. Host disease. For example, hydrophilic groups of synthetic antigens can be altered or designed to bind to TCRs but not evoke a response, thus inhibiting T cell proliferation.

Further, T cells are proposed to mediate most forms of inflammatory arthritis. In particular, CD1-restricted T cells are thought to influence the development of T_{H}1 immune responses, and their dysregulation has been found in models of systemic lupus erythematosus, systemic sclerosis and diabetes mellitus. Prior to the discovery of CD1 protein function, there was no cellular mechanism to account for glycolipid-specific T cell responses that could directly mediate disease or provide help to glycolipid-specific B cells. Until now, the molecular basis of CD1-mediated glycolipid antigen presentation was not known, nor was it possible to develop a molecular model for recognition of self glycolipids by autoreactive T cells. The advantage of understanding the molecular events underlying, for example, CD1c-presentation of lipids to the TCR and the cellular basis of presentation of lipid autoantigens is that it defines the structures of compositions, such as antibodies, which can block CD1c-autoantigen presentation. Further, synthetic antigens can be employed as immunomodulatory lipids for the treatment of rheumatic diseases.

The following examples describe specific aspects of the invention to illustrate the invention and provide a description of the methods used to isolate and modify the antigens of the invention and to identify the binding of these molecules. The examples should not be construed as limiting the invention in any way.

All citations in this application to materials and methods are hereby incorporated by reference.

### EXEMPLIFICATION

### EXAMPLE 1

### Bacteria and Antigens

M. phlei, M. tuberculosis H37Ra, M. fortuitum, M. smegmatis and M. bovis BCG were cultivated in 7H9 medium (Difco) supplemented with 0.05% Tween 80 and 1% glucose, mannose or galactose. Organic extracts (1×) were made by shaking 7.5 mg of lyophilized bacteria per 1 ml chloroform: methanol (2:1) at 20° C. for 2 hrs. Sonicates (1×) were made by probe sonication of 10 mg bacteria per 1 ml phosphate buffered saline, subsequently clarified by centrifugation as described by E.M. Beckman, et al. (1996) J. Immunol. 157:2795. Mycolyl glycolipids were purified using preparative silica TLC in solvent A (60:16:2 chloroform:methanol:water) and extraction from silica into chloroform:methanol (2:1) or by eluting an open 2×20 cm silica gel column serially with chloroform and acetone in a stepwise gradient. The antigenic glycolipid eluted at 30% acetone in chloroform.

The purified antigenic glycolipid was hydrolyzed and partitioned between aqueous and organic phases as described in E. M. Beckman, et al. (1994) Nature 372:691. Organic soluble products were derivatized with phenacyl bromide and coeluted on C18 reverse phases HPLC with M. tuberculosis mycolic acids. Beckman, 1994, supra. The carbohydrate structure was determined by methylating the reducing end of the intact glycolipid (0.5 N HCL in methanol at 65° C. for 2 hr), followed by alkaline hydrolysis. Aqueous phase products were acetylated and compared to acetylated methyl glycosides of authentic glucose and other carbohydrates by gas chromatography.

M. phlei, M. tuberculosis (Sigma) and synthetic (Ribi) α,α′-trehalose dimycolate were hydrolyzed to yield GMM by drying on glass and treating with 2M TFA at 121° C. for 2 hrs (G.S. Besra, et al. (1994) Proc. Nat. Acad. Sci., USA 91:12737). The yield of the resulting glycolipids was characterized by TLC in comparison with authentic GMM standards. ESI-MS analysis revealed ions of the expected m/z for GMM.

### EXAMPLE 2

### T Cell Lines and Assays

LDN5 was derived from the same human leprosy skin lesion that gave rise to the previously described LAM reactive T cell line LDN4 (P. A. Sieling, et al. (1995) Science 269:227). Cultures were stimulated initially with autologous GM-CSF and IL-4 treated CD1⁺ monocytes and M. leprae sonicate. After establishment of LDN5, cultures were maintained in IL-2 supplemented medium and periodically stimulated with allogenic CD1⁺ APCs and M. phlei sonicate (X/1000) containing GMM. FACS analysis of LDN5 revealed positivity for αβ TCR, but not CD4 or CD8β. T cell culture methods, proliferation assays and cytolysis assays were carried out according to the methods described in E. M. Beckman, et al. (1996) J. Immunol. 157:2795. All bioassays were done in triplicate and reported as mean +/−standard deviation.

All references cited are herein incorporated by reference.

Equivalents

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize or be able to ascertain using no more than routine experimentation, many equivalents to the specific embodiments of the invention described specifically herein. Such equivalents are intended to be encompassed in the scope of the claims.

What is claimed is:

1. A method for inducing a CD1-restricted T cell response which comprises administering to a mammal a synthetic antigen comprising one or more branched or unbranched acyl chains which bind to a CD1 protein and a hydrophilic moiety which is recognized by a T cell.

2. The method of claim 1 wherein one or more of the acyl chains has a length of about C₁₂ to greater than C₁₀₀.

3. The method of claim 2 wherein one or more of the acyl chains has a length of C₃₀ to C₉₀.

4. The method of claim 3 wherein one or more of the acyl chain is covalently bound to a phosphate group.

5. The method of claim 1 wherein the hydrophilic moiety is a carbohydrate.

6. The method of claim 1 wherein the composition is administered parenterally.

7. The method of claim 1 wherein the composition is administered mucosally.

8. The method of claim 1 wherein the hydrophilic moiety to which the T cell response is induced is selected from a viral, bacterial, fungal, parasitic, or tumor antigen.

9. The method of claim 1 further comprising one or more of the following components:
a) an adjuvant;
b) a peptide; or
c) an additional antigen.

10. A method for treating a disease in a mammal comprising administering to the mammal a synthetic composition which induces a CD1-restricted immune response to a hydrophilic component of the composition associated with the disease, wherein the hydrophilic component is conjugated to a hydrophobic component which comprises one or more saturated or unsaturated acyl chains.

11. The method of claim 10 wherein the hydrophilic component is selected from a viral, bacterial, fungal, parasitic, or tumor antigen.

12. The method of claim 11 wherein the disease is caused by a bacterium.

13. The method of claim 10 wherein the hydrophilic component is an autoimmune antigen.

14. The method of claim 10 wherein the composition is administered parenterally or mucosally.

15. The method of claim 10 wherein one or more of the acyl chains has a length of about C₁₂ to greater than C₁₀₀.

16. The method of claim 15 wherein one or more of the acyl chains is covalently bonded to a phosphate group.

17. The method of claim 10 wherein the hydrophilic component is a carbohydrate.

18. The method of claim 10 further comprising one or more of the following components:
a) an adjuvant;
b) a peptide; or
c) an additional antigen.

19. A method for inducing a CD1-restricted T cell response in a mammal comprising administering to the mammal an immunomodulating composition comprising a hydrophobic moiety which binds to a CD1 protein and a hydrophilic moiety which comprises an antigen recognized by a CD1-restricted T cell thereby inducing a CD1-restricted T cell response to the antigen.

20. The composition of claim 19 further comprising one or more of the following:
a) an adjuvant;
b) a peptide; or
c) an additional antigen.

21. A method for inducing a CD1-restricted T cell response which comprises administering to a mammal a synthetic antigen comprising one acyl chain which binds to a CD1 protein and a hydrophilic moiety which is recognized by a T cell.

22. The method of claim 21 wherein the acyl chain is covalently bound to a phosphate group.

23. The method of claim 22 wherein the β and γ carbons of the acyl chain are saturated.

24. The method of claim 22 wherein the β and γ carbons of the acyl chain are unsaturated.

25. The method of claim 21 wherein the acyl chain has a length of about C₁₂ to greater than C₁₀₀.

26. The method of claim 21 wherein the hydrophilic moiety is a carbohydrate.

27. The method of claim 21 wherein the composition is administered parenterally.

28. The method of claim 21 wherein the composition is administered mucosally.

29. The method of claim 21 wherein the hydrophilic moiety to which the T cell response is induced is selected from a viral, bacterial, fungal, parasitic, tumor or self antigen.

30. The method of claim 21 further comprising one or more of the following components:
a) an adjuvant;
b) a peptide; or
c) an additional antigen.

31. A method for modulating a CD1-restricted T cell response which comprises administering to a mammal a synthetic antigen comprising one branched acyl chain comprising a free mycolate which is recognized by a T cell.
